(19) United States
(12) Patent Application Publication
(10) Pub. No.: US 2012/0203069 A1
(43) Pub. Date: Aug. 9, 2012

(54) SURGICAL SHIELD FOR SOFT TISSUE PROTECTION

(76) Inventors: Blake Hannaford, Seattle, WA (US); Randall A. Bly, Seattle, WA (US); James S. Pridgeon, Seattle, WA (US); Eugene G. Chen, Carlsbad, CA (US); Kristen S. Moe, Seattle, WA (US); Louis Kim, Seattle, WA (US); Jacob Rosen, Santa Cruz, CA (US)

(21) Appl. No.: 13/369,952

(22) Filed: Feb. 9, 2012

Related U.S. Application Data
(63) Continuation-in-part of application No. 12/943,779, filed on Nov. 10, 2010.
(60) Provisional application No. 61/346,476, filed on May 20, 2010.
(60) Provisional application No. 61/293,932, filed on Jan. 11, 2010.
(60) Provisional application No. 61/261,310, filed on Nov. 14, 2009.

(51) Int. Cl. A61B 1/32

(52) U.S. Cl. 600/201

(57) ABSTRACT

A soft tissue protection surgical shield protects collateral soft tissue from damage during a surgical procedure within a surgical space of a body. The shield comprises an elongated flexible shield having a proximal end and a distal end. The proximal end has a first opening and the distal end has a second opening. The shield further comprises a side wall between the proximal and distal ends that defines the first and second openings. The side wall is conformal to the surgical space and arranged to resist perforation by surgical instruments in use during the surgical procedure, and also to define and maintain the access pathway to the surgical site.

### PRIORITY CLAIM

This application is a Continuation-in-Part of U.S. patent application Ser. No. 12/943,779 filed on Nov. 10, 2010 and now pending, which claims priority to U.S. Provisional Patent Application Nos. 61/261,310, filed Nov. 14, 2009; 61/293,932, filed Jan. 11, 2010; and 61/346,476, filed May 20, 2010; each incorporated herein by reference. U.S. Provisional Patent Application No. 61/______ filed on Feb. 9, 2012, Docket No. 79628.8001.US00, is also incorporated herein by reference.

### BACKGROUND OF THE INVENTION

Endoscopic surgery within the head is a common procedure in neurological surgery and otolaryngology. It is typically performed using a transnasal or sublabial route, but also can be carried out using a small eye-lid crease or conjunctival incision for a transorbital route. There are several advantages to endoscopic surgery of the brain, skull base and nasopharynx. It avoids large cranial incisions and bony openings, which require much more extensive exposures, brain retraction and wound healing. It also provides improved illumination and visualization of the target tissues because the camera of the endoscope is brought directly to the surgical site. Endoscopic surgery also permits target tissue treatment through small exposures and openings to the skull.

During this type of surgery, there tends to be some local trauma to the nasal mucosa, turbinates, nasal septum, and sphenoid/frontal/maxillary sinus, and, in the case of transorbital approaches, orbital and periorbital tissue. This surgical pathway trauma can add to the trauma of the procedure and prolong the patient's recovery time. In addition, there is frequent and persistent “run down” of mucous, blood, and soiled irrigation fluid that obscures the view of the endoscope. This leads to the constant need for irrigation and suction of the offending liquids, as well as the outright removal, cleaning and replacement of the endoscope. This can occur dozens of times during a single procedure, making the cleaning and clearing of the endoscope both time consuming and frustrating to the surgeon.

Accessing the surgical site through any route, but especially through either a transnasal or transorbital route, may require the surgeon to travel around or through internal tissue structures within the head. This can be extremely time consuming. For more complex procedures, an additional surgeon is sometimes called in specifically to access the surgical site. Whenever an instrument needs to be substituted, or an endoscope needs to be cleaned, the surrounding tissues are again put at risk as the instruments are removed and reinserted.

Therefore, there is a need to reduce or eliminate these aspects of endoscopic surgery, reduce soft tissue trauma, shorten operative times, and potentially lead to improved patient outcomes.

### SUMMARY OF THE INVENTION

A surgical shield protects collateral soft tissue from damage during a surgical procedure within a surgical space of a body. The shield may be provided as an elongated flexible sheath. The shield may be compressed, folded or invaginated so that it has relatively small diameter, during placement and/or removal. The shield may expand or unfold after it is at a desired position, providing a relatively large passageway or lumen for passage of an endoscope and surgical instruments. The shield may have one or more thin flexible sidewalls that can conform to the tissue around or bearing on the shield. Other areas or sidewalls of the shield may be thicker to better resist perforation by surgical instruments, and/or to better maintain the access lumen of pathway to the surgical site.

Other features and advantages will become apparent from the following detailed description of examples of how the invention may be designed and used. The invention resides as well in sub-combinations of the elements and method steps described.

### BRIEF DESCRIPTION OF THE DRAWINGS

In the drawings, the same element number indicates the same element in each of the views:

FIG. 1 is sectional view of a human head and surgical shield deployed in a nostril of the human head;

FIG. 2 is a sectional view, similar to FIG. 1 illustrating a condition of the shield when first introduced into the nostril of the human head;

FIG. 3 is a cross-sectional view taken along lines 3-3 of FIG. 1, illustrating a pair of shields within respective nostrils;

FIG. 4 is sectional view of a human head and another surgical shield deployed in a nostril of the human head;

FIG. 5 is sectional view of a human head and still another surgical shield deployed in a nostril of the human head;

FIG. 6 is a perspective view of another shield having texturing on an outer surface;

FIG. 7 is a sectional side view of a shield having an irrigation system;

FIG. 8 is a sectional side view of a shield having a suction system;

FIG. 9 is a sectional side view of a shield having perforated cutouts in a sidewall;

FIG. 10 is a sectional side view of a shield having a radio opaque material;

FIG. 11 is a sectional view similar FIG. 3 illustrating a pair of shields within nostrils and each including magnets for retaining the shields in place during a surgical procedure;

FIG. 12 is a sectional side view of a shield having optical fibers within the side wall;

FIG. 13 is a sectional side view of a shield having optical fibers and an irrigation system within the sidewall;

FIG. 14 is a side view, partly in section, illustrating a shield being deployed;

FIG. 15 is a side view, partly in section, illustrating the shield of FIG. 14 after deployment;

FIG. 16 is an end view of a shield having internal passageways for irrigation or suction or illumination.

FIG. 17 is a perspective view of the shield shown in FIG. 16.

FIG. 18 is a perspective view of a shield having internal passageways with in-facing ports, for irrigation or suction, or with in-facing lighting elements.

FIG. 19 is an end view of the shield shown in FIG. 18.

FIG. 20 is a diagram of a shield having back-to-back conical sections.

FIG. 21 is a view of a distal end of the shield of FIG. 20 folded or pressed flat.

FIG. 22 is an end view of a distal end of the shield of FIG. 20 in a rolled configuration.

FIG. 23 is an end view of a distal end of the shield of FIG. 20 having an invagination.

FIGS. 24 and 25 are side views of instruments for positioning and/or deployment of the shields shown in the other figures.

FIG. 26 is an end view of a distal end of the shield of FIG. 20 having multiple invaginations.

FIG. 27 is a side view of an assembly including a shield, an applicator or tamping tool.

### DETAILED DESCRIPTION

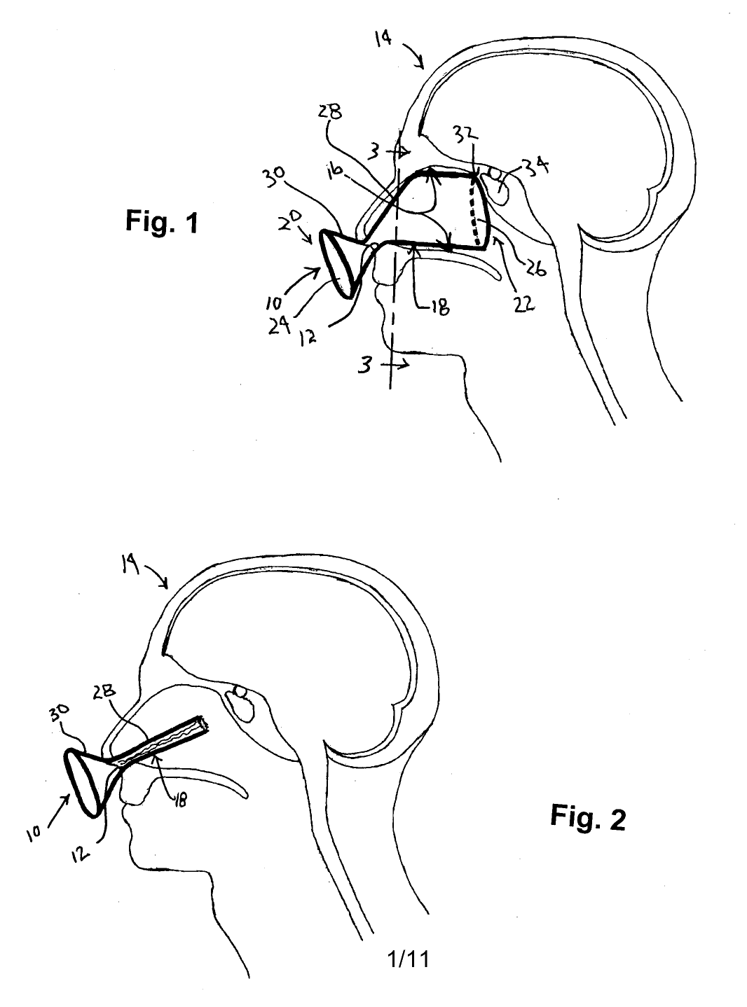

FIG. 1 shows a surgical shield 10 deployed within a nostril 12 of a human head 14. The shield provides a collateral soft tissue protection surgical shield that protects collateral soft tissue from damage during a surgical procedure within a surgical space 16 of a body. The shield generally includes an elongated flexible sheath 18 having a proximal end 20 and a distal end 22. The proximal end has as a first opening 24 and the distal end has a second opening 26. A sidewall 28 between the proximal end 20 and the distal end 22 defines the openings 24 and 26. The sidewall 28 conforms to the surgical space 16 and arranged to resist perforation by surgical instruments in use during the surgical procedure, and also to define and maintain the access pathway to the surgical site. The shield may be formed of, for example, latex rubber, silicone rubber, latex or polymeric silicone substances, or other flexible polymer materials and/or other biocompatible elastic material. In FIGS. 1, 2 and 5, the head of the patient is shown in an upright orientation, of purpose of illustration. In actual use, with the patient on an operating table, the head of the patient is facing up, rather than to one side as in these Figures.

The shield 10 has a generally conical or tapering section 30 at its proximal end 20 that extends proximally from the nostril 12. The tapering section 30 together with the tapered side wall 28 serve to maintain the shield in place during the surgical procedure. The horn shape 30 also permits instruments to be deployed through a wide angle, range to fully address the surgical site 32. The surgical site may, for example, include a lesion 34 in need of removal.

The shield 10 is collapsible and can be folded in various ways. The opening in the body leading to surgical portal may be smaller than the portal itself. Thus, it can be necessary to temporarily decrease the diameter of the shield before moving it into position. With the internal and external flare or conical section designs, the external flare prevents the shield from moving forward as the flare rests against the vestibule of the nares. Internally, the shield also flares outwardly, which helps to prevent the shield from moving backward. When used in the nose, the internal conical section can rest against the nasal bones and cartilage to prevent rearward movement. The conical or flared out sections held to hold the shield secure when instruments are passed in and out.

FIG. 2 shows how the shield 10 may be deployed in the nostril 12 of the patient's head 14. However, the shields of the present invention may be used to advantages in other surgical approaches as well including transorbital approaches or conjunctival incisions. Here it may be seen that the sidewall 28 of the shield 18 is in a collapsed state distally from the horn shaped portion 30. This enables ready insertion of the shield 18 into the nostril 12. Once the shield 18 is positioned within the nostril 12 as illustrated, the shield 18 may be expanded to conform to the surgical space. To that end, the shield may have a natural full shape to which it naturally expands from a compressed condition once it is released.

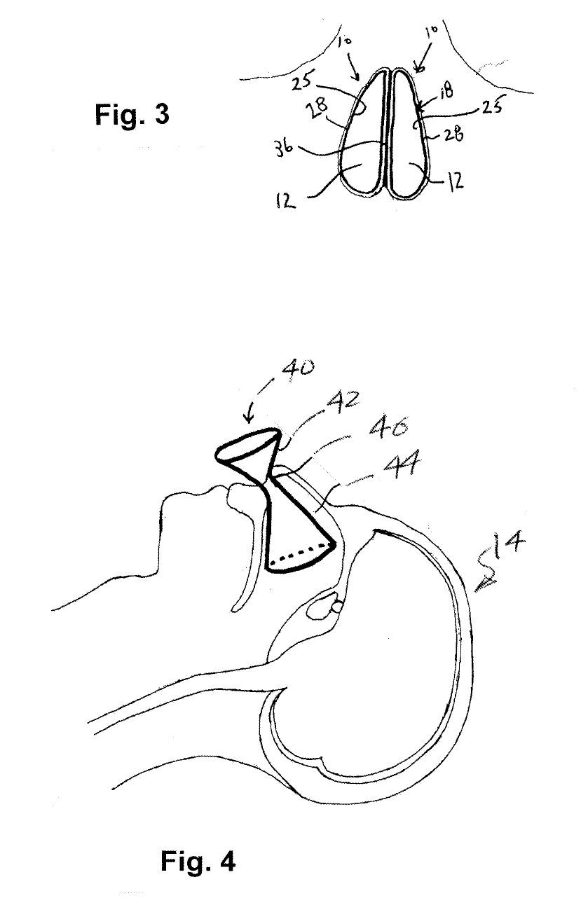

When expanded from the low profile shape as seen in FIG. 2 to the expanded shape, the shield 18 will conform to the surgical space. This may be seen in the sectional view of FIG. 3. Here, two identical shields 10 are deployed on either side of the septum 36. The sidewalls 28 of the shield 10 are fully conformed to the inner wall of the nostrils 12. By conforming to the inner wall of the nostrils 12, the shields 10 also define and maintain the access pathway to the surgical site. In addition, each shield 10 has an inner surface 25. The inner surface 25 may be coated with a low friction coating. Suitable coating materials include, for example, PTFE, hyaluronan, and glycerin. This makes the inner surface a low friction surface to assist in easier insertion of instruments into the shield 18 and avoiding piercing the shield and the collateral soft tissue with the instruments.

Generally, the inner surface of the shield is smooth, to better allow sliding of instruments and endoscopes along the inner surface, without moving shield. The outer surface may be hydrophilic and have a tendency to adhere to the mucosa, also to reduce inadvertent movement of the shield. The shield may be transparent or translucent, so that the mucosa and anatomical landmarks may be viewed through the shield via an endoscope.

Referring still to FIG. 2, the shield 18 requires a degree of stiffness so that it may be guided into position without buckling, curling, or excessively deflecting from the desired path. The shield 18 may be sufficiently stiff simply as a result of the material selection and material thickness. Alternatively, one or more stiffening elements 51, such as rods, wires, or plates may be attached to, or integrally formed with, the shield to provided stiffness.

FIG. 4 illustrates another shield 40 having an hour glass shape, including a flared proximal portion 42 and a flared distal portion 44. A reduced dimension section 46 joins the portions 42 and 44 and serves to hold the shield 40 in place during the surgery. The shield 40 may be deployed in the same manner as previously described with respect to the shield 10 of FIG. 1.

In some designs, the shield may automatically expand or unfurl from the rolled up or compressed position shown in FIG. 2 into a fully deployed position as shown in FIGS. 1 and 4, via a natural spring force of the material. The surgeon may assist in the unfurling, if necessary, by selectively pushing on the shield using a surgical tool. The shield may alternatively include one or more spring elements 48 attached onto or embedded into the shield, with the spring elements exerting an unfurling force on the shield 18. The spring elements, if used, may help to unfurl the shield 18 or distal portion 44, and to hold it open against the surrounding tissue. The spring elements 48 may also be adapted hold the open shield into a desired shape.

The shields 10 and 40 may be provided in varying lengths and diameters, with the surgeon selecting a specific size based on the specific anatomy of the patient, or other factors. The shields may also be used in methods where they are cut to a desired length by the surgeon, prior to placement within the nostril, or other opening. As shown in FIG. 4, scale markings 45 may be printed or molded onto the shields to assist the surgeon in cutting the shield to the desired size. In some cases, it may also be useful to cut the horn shape 30 or proximal portion 42 to a desired length and/or shape, before or after the shield placed.

In some cases, leaving the shield in place for e.g., 7 days after surgery may help in healing process. Consequently, it may be desirable to shorten or minimize the horn shape or proximal portion of the shield, even after surgery.

In FIG. 4, the conical portions on opposite sides of the narrow section 46 may be sufficient to hold the shield in place. Alternatively, or in addition, the proximal portion 42 may temporarily attached to the patient's nose via suture or a clip. As shown in dotted lines in FIG. 4, an inflatable balloon 49 may also be included with the shield to help hold the shield in place. If used, the inflatable balloon 49 may also help to block fluids from draining into the airway.

Referring to FIGS. 1, 2 and 4, the shield may be removed by turning it in the roll-up or furl direction, to return the shield back to or near its original configuration as shown in FIG. 2. This may be achieved by grasping and turning the proximal portion, by hand or using a tool.

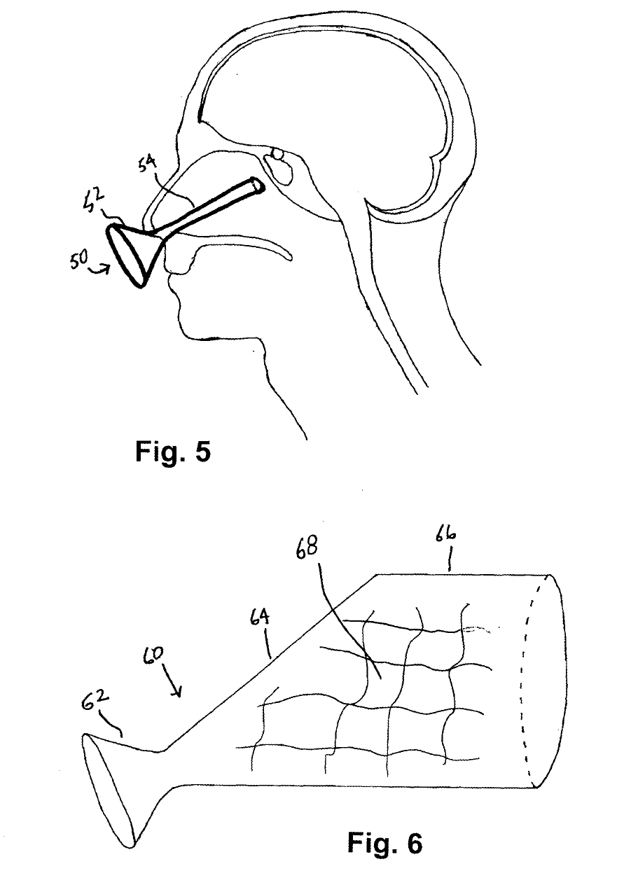

In FIG. 5, a shield 50 is horn shaped in a proximal portion 52 and elongated in distal portion 54. This shield may be used to advantage when the surgical target of relatively small size, not requiring surgical instruments to be deployed through a wide angle range to fully address the surgical site.

FIG. 6 shows another shield 60 similar to the shield 10 of FIG. 1 and having a horn shape 62 at its proximal end and a tapered shape 64 that leads to a cylindrical shape 66 at its distal end. The shield 60 may be formed from any of the materials previously mentioned. The shield 60 further has a textured sidewall 68. The textured sidewall 68 provides a gentle friction with the collateral soft tissue to assist in maintaining the shield in place. The texturing may be included in shields of any shape including the horn or hourglass configurations disclosed herein.

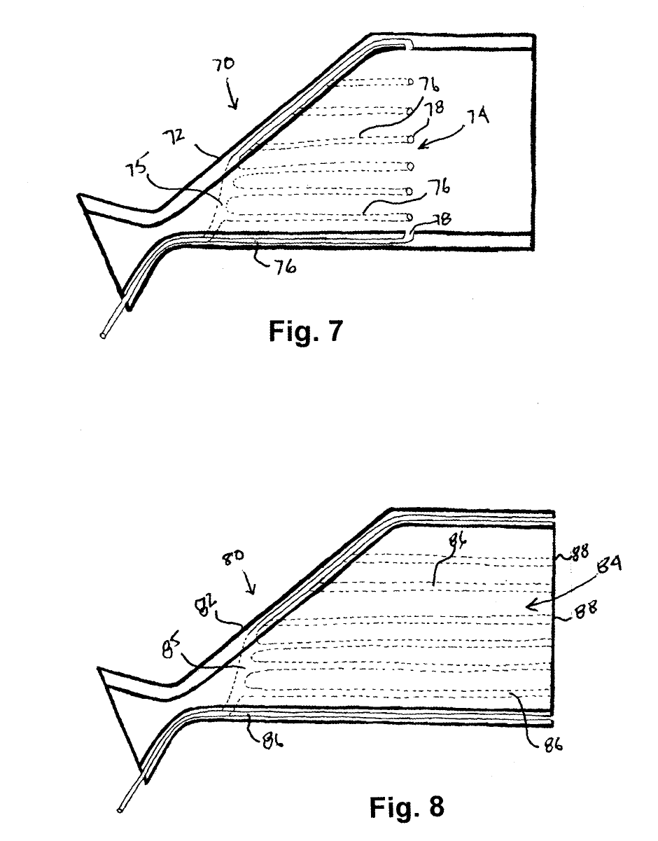

In FIG. 7 another shield 70 is shaped like the shield 10 of FIG. 1 and can be formed from the same materials previously mentioned. Here however, the shield includes an irrigation system 74 within its sidewall 72. More specifically, the sidewall has an internal feed channel 75 that communicates with internal distribution channels 76. The distribution channels terminate at ports 78 to admit cleaning solution, such as saline solution, for example, into the surgical site. As a result, the surgical site may be cleaned without the need for the removal of surgical instruments, such as an endoscope, from the surgical site. The endoscope may also be used without any irrigation shield, since the endoscope may be irrigated by the irrigation system 74 in the shield 70. The endoscope diameter can then be effectively reduced by up to about 50%. This allows for greater flexibility in performing surgery. As may be appreciated, the irrigation system could also be included in shields having the horn or hourglass configurations as well.

FIG. 8 illustrates another shield 80 also shaped like the shield 10 of FIG. 1 and can be formed from the same materials previously mentioned. Here however, the shield includes a suction system 84 within its sidewall 82. More specifically, the sidewall has an internal common channel 85 that communicates with internal branch channels 86. The branch channels extend all of the way to the end of the shield 80 and terminate at ports 88. The extended branch channels 86 render the shield capable of providing suction for removal of fluids such as “run down” of blood, mucous, and soiled irrigation fluid that may obscure endoscopic visualization. Of course, the suction system could also be present on the shields having the horn or hourglass configurations as well.

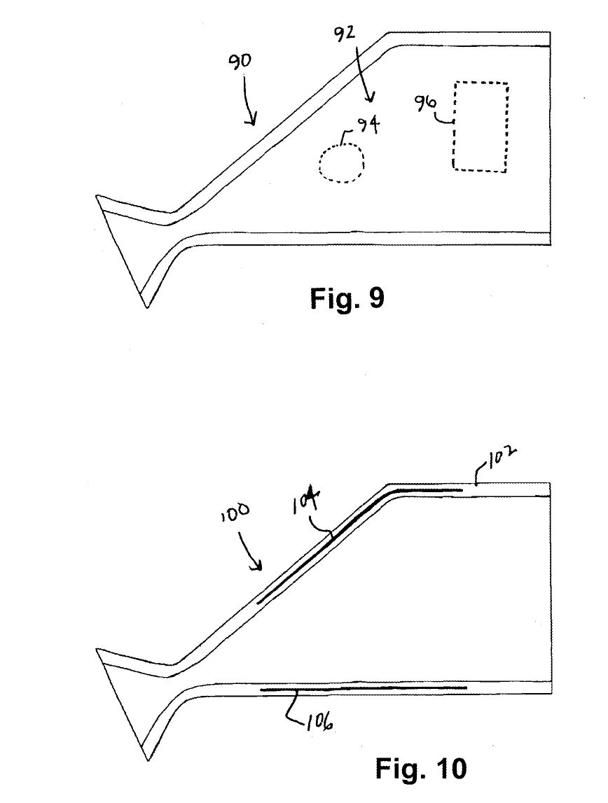

FIG. 9 illustrates another shield 90 also shaped like the shield 10 of FIG. 1 and can be formed from the same materials previously mentioned. Here however, the shield includes perforated cutouts 94 and 96 within the sidewall 92 of the shield 90. The cutouts assist in removing portions of the sidewall 92 should it be necessary to permit collateral projecting tissue to extend there through. This not only facilitates retention of the shield, but also potential removal of the projecting tissue should that be necessary. As may be appreciated, the cutouts could also be present on any of the disclosed embodiments herein including the shields having the horn or hourglass configurations. The cutouts may be removed by grasping with a tool and pulling them out. In some cases where the need for sidewall openings is known in advance, one or more cutouts may be removed from the shield before it is placed into the patient.

FIG. 10 illustrates another shield 100 also shaped like the shield 10 of FIG. 1 and can be formed from the same materials previously mentioned. Here however, the shield includes radio opaque material 104 and 106 within the sidewall 102 of the shield 100. Since the radio opaque material 104 and 106 is within the sidewall 102 of the shield 100, and since the sidewall conforms to the shape of the surgical space, the margins of the surgical space will be clearly visible under fluoroscopy during a surgical procedure. The radio opaque material will also make the presence of the shield 100 obvious under fluoroscopy to assist in guarding against the potential for the shield 100 to be left in the patient after the surgical procedure is completed.

The radio opaque material may also incorporated into any of the shields described here, including the shields having the horn or hourglass configurations. The radio opaque material may be strips, wires, dots, or other shapes of metal material. A radiolucent strip embedded in the walls of the shield allows for confirmation of placement with fluoroscopy, and it also may be registered and integrated with surgical navigation. Multiple strips may make registration and/or orientation more convenient. The strip could be embedded in any wall of the device.

Referring still to FIG. 10, strips or wires 104 and/or 106 may also be a malleable material, such as a copper wire, which acts as a shape holding element. One or wires or strips in or on the shield may be used to allow the surgeon to bend the shield into a specific shape or configuration, with the strips 104 or 106 then holding the shield in that shape. In this case, the strips or wires are thick enough to prevent the e.g., plastic or rubber material of the shield from reverting back to its original shape.

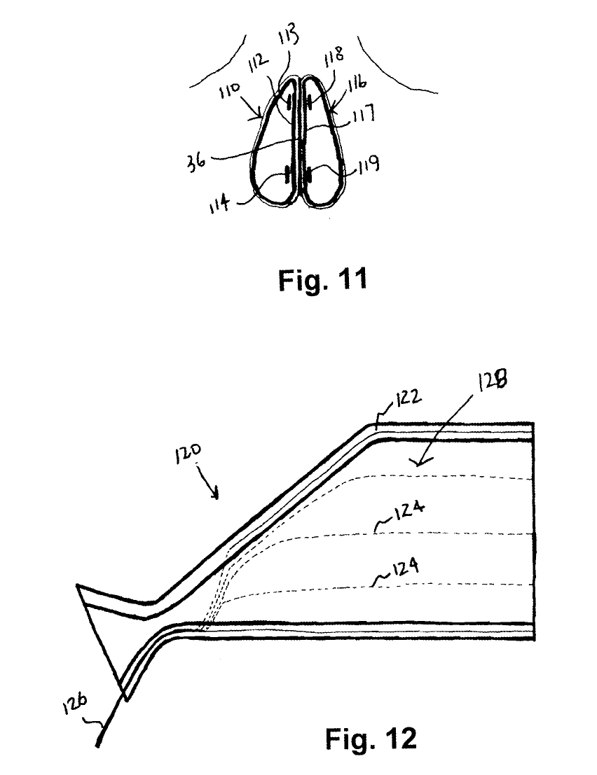

FIG. 11 is a sectional view similar to the sectional view of FIG. 3. Here it may be seen that a pair of shields, shields 110 and 116 have been deployed on opposite sides of a septum 36. Shield 110 has sidewall 112 and shield 116 has sidewall 117. Sidewall 112 carries magnets 113 and 114 and sidewall 117 carries magnets 118 and 119. The magnets are positioned so that magnet 113 is opposite magnet 118, and magnet 114 is opposite magnet 119. The attraction between the magnet pairs serves to gently hold the shield 110 and 116 in place during the surgical procedure employing the shields 110 and 116.

FIG. 12 illustrates another shield 120 embodying further aspects of the also shaped like the shield 10 of FIG. 1 and can be formed from the same materials previously mentioned. Here however, the shield includes a light projection system 128 within its sidewall 122. More specifically, the sidewall 122 has an internal common optical fiber 126 that serves as a light source and is coupled to internal branch optical fibers 124. The branch optical fibers 124 extend all of the way to the end of the shield 120. The extended optical fibers 124 render the shield capable of projecting light from the end of the shield 120 onto the surgical site. This supports visualization of the surgical procedure. Light for the common source 126 may be obtained from a light emitting diode or other source known in the art. As may be appreciated, the light projection system could also be employed in any of the shields disclosed herein, including the shields having the horn or hourglass configurations. One or more of the optical fibers 124 may be connected to a lens 125 at leading or distal end of the shield 128, to provide an imaging capability. For certain procedures, this design may obviate the need for using an endoscope.

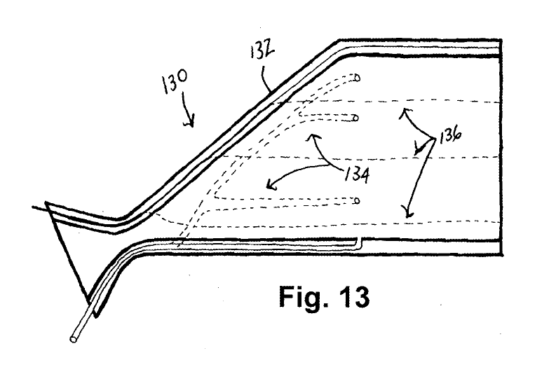

FIG. 13 illustrates another shield 130 embodying further aspects of the also shaped like the shield 10 of FIG. 1 and can be formed from the same materials previously mentioned. Here however, the shield includes a combination irrigation system 134 and light projection system 136. The irrigation system 134 and light projection system 136 are formed in the sidewall 132 in the same manner as previously described. The combination irrigation system and light projection system may also be included in any of the shields disclosed herein, including the shield having the horn or hourglass configurations.

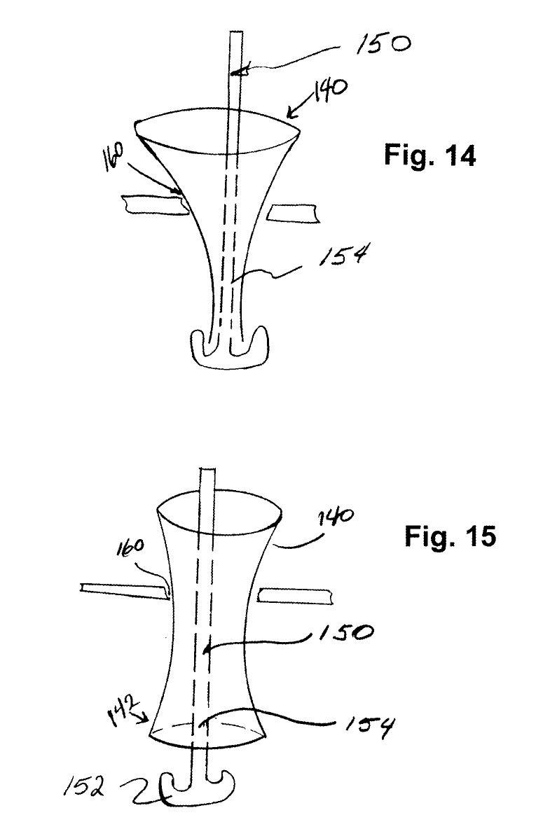

Referring now to FIGS. 14 and 15, a shield 140 has an hour glass shape and may be formed from a soft and flexible material, such as rubber. In FIG. 14, the leading or distal end 142 of the shield 140 is gathered and collapsed held into a cylinder end 152 on the shaft 154 of a deployment tool 150. The shaft 154 of the deployment tool 150 is flexible so that it can bend and deflect as may be needed to advance the shield and the tool 150 through and/or past tissue and passageways, between the entry way, for example, a nostril, and the surgical site, for example, the pituitary gland in the head. The shaft 154 is also designed to resist buckling, so that nominal compressive loading on the shaft, for example when the cylinder 152 pushes against or past tissue, do not cause the shaft to excessively deflect. In one design, the shaft 154 may include a wire coil, optionally within a flexible plastic or rubber tube, or having a flexible coating.

The shaft 154 of the deployment tool 150 is inserted into an opening 160 in the skull, for example, a nostril. The shaft 154 is advanced to position the shield 140 as desired. The shaft is then further advanced while the shield 140 remains stationary. The end 142 of the shield 140 expands to its full configuration. The tool 150 is then removed through the shield 140 to complete the shield deployment.

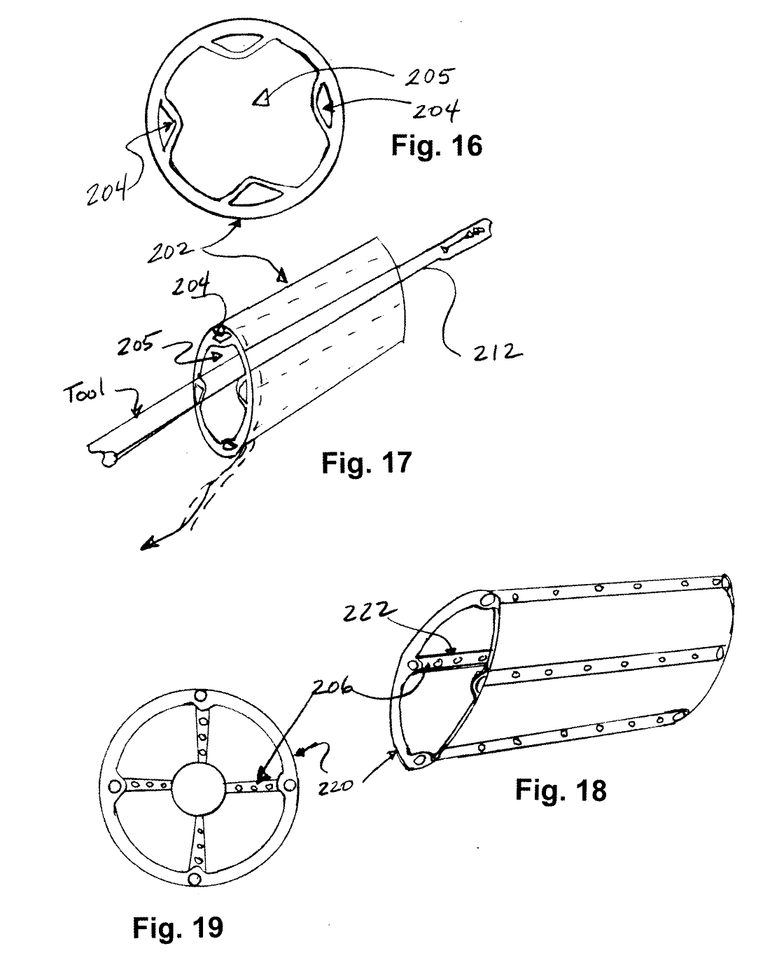

Turning to FIGS. 16 and 17, a shield 202 has internal passageways 204 separated from the main passageway 205 by internal walls 206. The passageways 204 may be integral with the shield 202, or molded into the shield. Although the passageways 204 are shown as generally triangular, other shapes, sizes, numbers and arrangements of passageways may be used. A manifold 208 at the back or proximal end of the shield 202 may be provided to connect each of the passageways 204 to a single irrigation supply line or aspiration line 210. The manifold 208 may also be integral with the body of the shield 202. The supply line or aspiration line 210 connection to the manifold 208 is on one side of the shield 202. This leaves the main passageway 205 of the shield unobstructed, to allow instruments to be passed through and removed from the main passageway 205 without interference. The internal walls 206 are typically flexible or compressible. This also helps to allow passage of instruments through the shield 202.

FIGS. 18 and 19 show a shield 220 which may be the same as the shield 202 shown in FIGS. 16 and 17, but with perforations or openings 222 in the internal walls 206. The openings 222 allow the inside surfaces of the shield 220 to be continuously or intermittently rinsed, or evacuated via vacuum. Alternatively the openings may be replaced with LEDs or other lighting elements, to illuminate the inside of the shield. If the shield is made of a transparent or translucent material, the tissues surrounding the shield may also be illuminated by the lighting elements. It is also possible to position lighting elements to direct light outwardly from the external surfaces of the shield walls, rather than internally as in FIGS. 18 and 19.

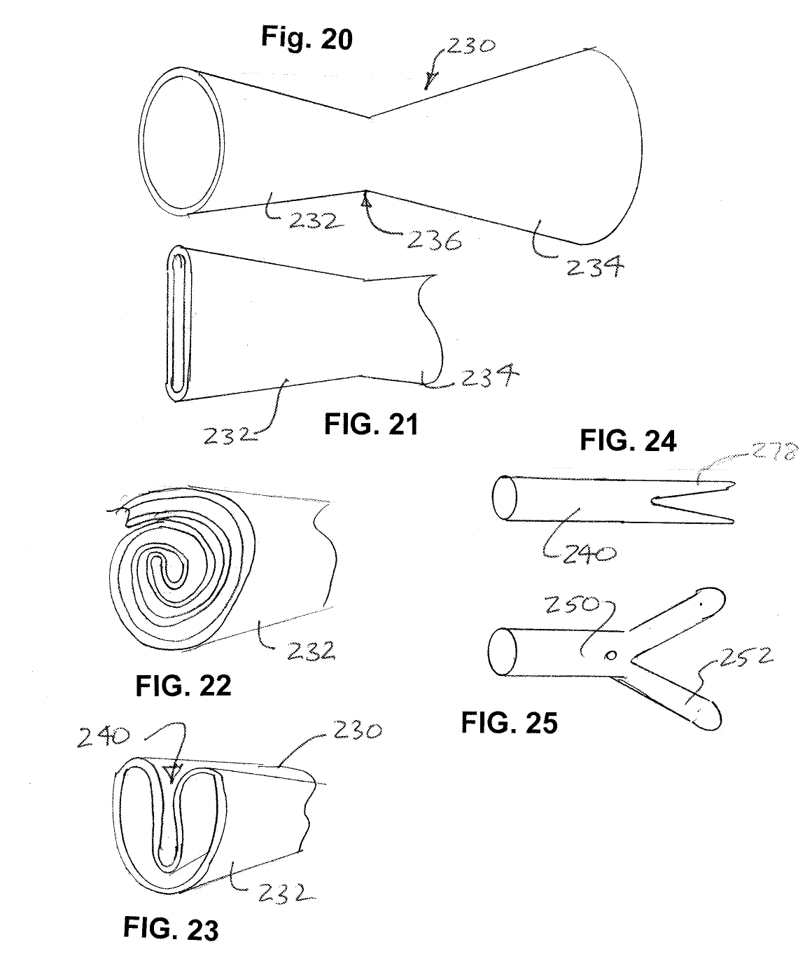

FIGS. 20-23 and 26 illustrate options for reducing the diameter of any of the shields described above, so that the shield may fit through a narrow opening in the body. These figures show the distal end of the shield. In many or most procedures, the proximal or back end of the shield remains outside of the body at all times. Since the proximal end of the shield does not need to pass through any small diameter body opening, ordinarily there is no advantage in also placing the back end of the shield into a reduced diameter configuration.

FIG. 20 shows a shield 230 having a first conical section 232 joined to second conical section 234 at a minimum diameter intersection 236. The sections 232 and 234 can of course have other shapes, and conical shapes are shown as one example. As shown in FIG. 21, the shield 230 may be flattened, and then rolled up, as shown in FIG. 22. This greatly reduces the diameter of the shield. It can also be quickly and easily performed in the operating room, as needed. However, getting the shield 230 to unroll after it is placed may be difficult in some circumstances. A forked end tool 240 may engage the center or core area of the rolled up shield 230, and unwind or unroll the shield.

FIG. 23 shows the shield 230 temporarily formed with an invagination 240. This also reduces the diameter of the shield 230 during placement. However, after the shield is placed, the invagination may be readily reversed, with the shield returning to its full diameter. Unlike the rolled configuration of FIG. 22, the invaginated configuration of FIG. 23 requires no positive unrolling to fully expand. The forked end tool 240 shown in FIG. 24 may be used to form and/or hold the invagination 240 in the shield 230. When the tool 240 is removed from the shield 230, the invagination 240 can reverse, or pop out, via the elastic characteristic of the shield material. If necessary, reversing the invagination may be assisted via pushing on it with an instrument inserted into the shield. For example, an expanding instrument 250 as shown in FIG. 25 may be inserted into the invaginated shield 230, with the arms 252 of the instrument 250 closed. The arms may then be opened, pushing the invagination 240 out, so that the shield 230 returns to its original expanded position.

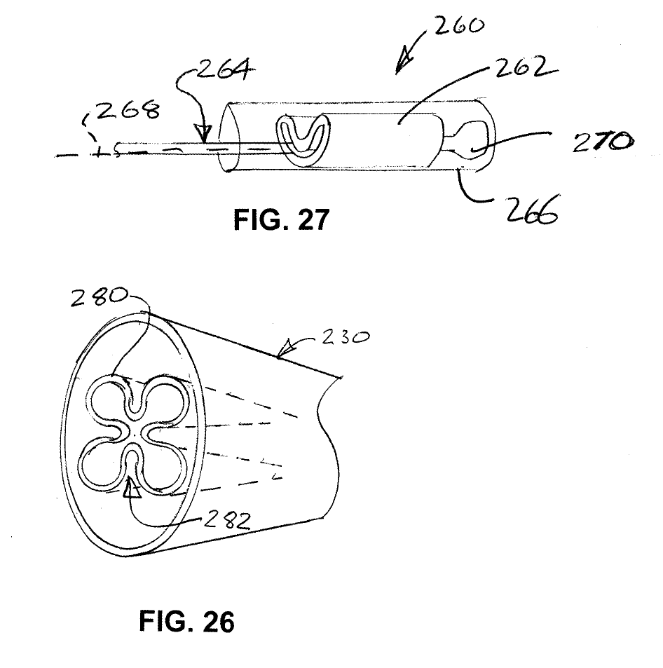

FIG. 26 shows a shield 230 placed into a reduced diameter configuration by providing multiple invaginations 280 (four in this example). The shield is also shown in the original or expanded configuration in FIG. 26, for comparison. A shield having multiple invaginations, as in FIG. 26, may be deployed using a forked end tool, as in FIG. 24, with the tool having tines 278 matched in number, size and orientation to fit in the creases 282 formed by the invaginations 280. Other techniques may also be used to put the shield into a reduced diameter configuration. For example, the shield may be folded accordion style. The shield may also be twisted into a spiral.

FIG. 27 shows shield delivery assembly 260 having a shield 262 which is folded, rolled, invaginated or otherwise temporarily reduced in diameter. A central, disposable, applicator 264 having a flexible and buckling resistant shaft is used to insert the shield 262. A disposable sheath 266 encases the shield 262 and keeps it in the reduced diameter configuration. Once inserted to appropriate depth, an actuator 268 is triggered to release the shield 266. The actuator severs the connection between the sheath and the shield. This allows the shield 262 to expand to its natural state, filling the portal. The sheath 266 and rigid applicator 264 are then removed together.

As used here, reduced diameter, configuration means a configuration where the shield has a maximum width or height that is reduced to allow the shield to pass through a relatively smaller opening, as may be necessary to place the shield during surgery. Since the shield is not necessarily circular in each instance, the term diameter here includes the dimensions of non-round shapes as well. In other words, diameter here means the largest characteristic dimension of the shield that determines whether it can or cannot pass through a given opening.

In a modification of the design shown in FIG. 27, a soft foam ball or plunger 270 is provided on the front or distal end of the applicator 264. The applicator 264 may then be used for both routing and guiding the shield into position, and also as a tamping tool. The tamping tool is inserted through the shield and the shield is configured into a reduced diameter configuration, by folding, wrapping, invaginating, or other technique. The tamping tool, surrounded by the shield, is inserted into an insertion tube. The insertion tube may be similar to the sheath 266, but with sufficient wall thickness and stiffness to generally maintain it cylindrical shape during the insertion procedure. In contrast, the sheath 266 may be thin wall design with little or no practical stiffness, which acts only a membrane or wrapper over the shield. Unlike the disposable sheath 266, use of the design having the insertion tube requires no actuator to sever any component of the assembly.

The tamping tool, surrounded by the shield, and the shield within insertion tube, may be provided pre-assembled, for example as a sterile unit within a single package. The shield is placed using the tamping tool to manipulate and guide the assembly into place. The insertion tube holds the shield in the reduced diameter configuration. The insertion tube is then withdrawn, while the tamping tool holds the shield in place. The inside surfaces of the insertion sleeve may be coated with a lubricant, to allow the insertion sleeve to more easily slide rearward off of the shield.

Referring still to FIG. 27, an alternative design omits the sheath 266 and uses an applicator in the form of a padded tamping device 264 to deploy the shield 262, and to avoid tearing and improve positioning. The padded tamping device 264 may have a sponge end and a rigid shaft. The shield 262 may be wrapped or twisted around the tamping device 264, which can be used for both initial insertion (by following the direction of wrapping to maintain tightness), and also deploying/unfurling (by holding the proximal end of shield 262 in place and reversing the direction of the tamping device), and then positioning the shield 262, by using the tamping device to press against the interior passageway. In this design, the shield 262 may be packaged as a unit which includes the tamping/deployment device 264.

The shields may have a flap at the inside flare that is pushed out once at the surgical site; this would act as a ‘bridge’. The shield may have a thicker side to give it some shape and rigidity while a thinner side allows it to conform to the tissue/structures.

Table 1 below describes features and benefits that may be provided. Of course, not every listed benefit and feature is necessarily realized in all embodiments, and in all methods of use of the shields. Rather, Table 1 lists benefits and features that may or may not be realized, in varying degrees, depending on the specific shield design used, the configuration of the shield, the delivery devices, and the methods of use.

TABLE 1

| Benefits | Features |
|---|---|
| Directional conduit through the nasal | Shield bridges the frontal sinus to the |
| sinuses | sphenoidal sinus |
| | Shield bridges the nostril to the |
| | postopharynx and paranasal sinuses |
| Preventing blood and other fluids from | Shield diameter is larger than the |
| dripping down the instruments into the | operating “portal” . . . shield allows multiple |
| operating field | instruments to be used through the shield |
| | which are protected from fluids and is still |
| | large enough diameter to allow |
| | instruments to move freely. |
| Allowing blind passage of instrument | Scope should remain focused on |
| exchange | operating sight . . . shield allows scope to |
| | remain in place while instruments are |
| | taken in and out. Because the shield |
| | “points” toward the operating site, |
| | instruments are directed back to the |
| | operating site without visualization. |
| Providing blind passage of instrument | Same as above . . . two nostril approach or |
| exchange in other nostril without scope | any other access point |
| Directing instruments ‘blindly’ to the | Same |
| operating site through Sphenoid sinus | |
| Tamponading tissue to reduce bleeding | Device conforms to the tissue in the sinus |
| | to tamponade the bleeding sites that were |
| | just operated on to reach operating site. |
| Providing wide angle of viewing of | The device gets wider from the nostril |
| pituitary gland and other sensitive organs | opening to the operating site to allow |
| (optic nerve and carotid) | better viewing. |
| Allowing two instruments to be used | The device gets wider from the nostril |
| through one nostril in an easy fashion | opening to the operating site to allow |
| | instruments to not clash and to have room |
| | to operate at a distance from leverage |
| | point of nostril. |
| Providing viewing of sinus cavity | The device is clear to allow for viewing of |
| structures | the entire endonasal contents |
| Minimizes need to remove nasal septum | The device provides path for instruments |
| tissue and turbinates | that normally is made by removing |
| | tissue . . . less tissue potentially needs to be |
| | removed |
| Reduces operating time significantly (by | The device allows a clean operating field |
| roughly 15-20%) | and most importantly keeps the scope |
| (1 min of OR time = $10) | clean. The procedures take a great deal |
| Reduces surgeon frustration | of time because of the constant need to |
| | take the scope out, clean the scope and |
| | then try to keep the scope clean when |
| | reinserted |
| Increases operating exposure of | View |
| operating site | |
| Surgical Efficacy and Safety | |
| Sleeve remains in place during instrument | Device goes from large diameter funnel to |
| exchange | smaller diameter at nostril to larger |
| | diameter to hold in place |
| Stability of device during surgery | Material feature: smooth inside, friction on |
| | outside surface |
| Aiding in recovery, replaces existing nasal | Material feature: long term (several hour) |
| splint | biocompatability |
| Benefit of transmitting light to aide | Material feature: transparency |
| visualization | |
| Illuminates surgical field | |
| Benefit of easy to deploy, first time correct | Material/shape feature: rolls up/reduces |
| positioning, aide to nonexpert surgeons | diameter and returns to original shape |
| | quickly |
| Benefit of structure to deploy | Thick and thin sides |
| But flexible to adhere | |
| Expansion/deployment | Material feature: Nitonol (Nickel-Titanium) |
| | ring |
| Benefit of easier to insert instruments | Shape Feature: flare at proximal end |
| (without moving eyes down to look at the | |
| opening) | |
| Irrigation without blocking passage | Irrigation channels integrated into shield |
| Illumination of passageway | Optical fibers or LEDS are integrated into |
| | the shield |

From the foregoing, it can be seen that the invention provides surgical devices that protect collateral soft tissue from damage during a surgical procedures, and also define and maintain the access pathway to the surgical site. The shields may incorporate many different functions to assist in the surgery including irrigation, suction, and light projection. The shields are shaped to afford wide angle instrument use to address large surgical sites. By virtue of the present invention, soft tissue trauma is reduced, operating times are reduced, and improved patient outcomes are made possible.

While particular embodiments of the invention has been shown and described, changes and modifications may be made. It is therefore intended to cover in the appended claims all such changes and modifications which fall within the true spirit and scope of the invention.

1. A method of performing a surgical procedure through an orifice in the head a patient, comprising:
moving a distal end of a shield through the orifice;
securing the shield in position in the orifice, with a proximal end of the shield external to the head of the patient, and with shield having at least one flexible sidewall substantially conforming to the orifice;
moving a distal end of a surgical instrument through the shield and into the head of the patient;
performing a surgical procedure in the head of the patient using the surgical instrument; and
removing the shield from the orifice.

2. The method of claim 1 where the orifice is a nostril.

3. The method of claim 1 where the orifice is an incision made in the head of the patient.

4. The method of claim 1 with the shield comprising a flexible thin wall material that conforms to the orifice.

5. The method of claim 1 with the proximal end of the shield stiffer than the distal end of the shield.

6. The method of claim 1 with the shield comprising medical grade plastic or rubber.

7. The method of claim 6 with the shield having a material thickness ranging from 0.01 to 0.12 inches.

8. The method of claim 1 with the distal and/or proximal ends of the shield having a conical shape.

9. The method of claim 1 further comprising securing the proximal end of the shield to relative to the nose of the patient.

10. The method of claim 9 with the securing performed by inflating a balloon, clamping or suturing.

11. A method of performing a surgical procedure at a surgical site within a surgical space of a body while protecting collateral soft tissue from damage, comprising:
providing a protective device comprising an elongated flexible shield having a proximal end and a distal end, the proximal end having a first opening and the distal end having a second opening, the shield further comprising a side wall between the proximal and distal ends;
placing the protective device into the surgical space;
conforming the side wall of the protective device to the surgical space while exposing surgical site at the distal end of the protective device;
performing the surgical procedure; and
removing the protective device from the surgical space.

12. The method of claim 11 comprising leaving the device in the patient for a duration after the surgical procedure, to aid in healing.

13. The method of claim 12 wherein the protective device is expandable from a low profile shape to an expanded shape and wherein the placing step includes introducing the protective device within the surgical space in the low profile shape and thereafter expanding the protective device to the expanded shape to conform the side wall of the protective device to the surgical space after being introduced into the surgical space.

14. The method of claim 11 further including placing a lubricant on the exterior of the device prior to inserting it and a coating on the interior following insertion.

15. The method of claim 11 further including irrigating the surgical site via an irrigation channel on or in the protective device.

16. The method of claim 11 further comprising aspirating the surgical site via a suction channel on or in the protective device.

17. The method of claim 11 further comprising illuminating at least a portion of the surgical space using a light source on or in the protective device.

18. A method of performing a surgical procedure through an orifice in the head a patient, comprising:
providing a shield having a distal end in a reduced size configuration;
moving the distal end of the shield through the orifice;
allowing the distal end of the shield to expand to a full size configuration;
securing the shield in position in the orifice, with a proximal end of the shield external to the head of the patient, and with shield having at least one flexible sidewall substantially conforming to the orifice;
moving a distal end of a surgical instrument through the shield and into the head of the patient;
performing a surgical procedure in the head of the patient using the surgical instrument; and
removing the shield from the orifice.

19. The method of claim 18 further comprising maintaining the distal end of the shield in the reduced size configuration by providing a sleeve around the distal end of the shield, and removing the sleeve after the distal end of the shield is in position.

20. The method of claim 18 further comprising maintaining the distal end of the shield in the reduced size configuration by holding it within a delivery tube, and removing the delivery tube from the sleeve after the distal end of the shield is in position.

21. A shield for protecting soft tissue during endoscopic surgery, comprising:
a first section joined to a second section at a waist, with at least part of the first section tapering inward from a proximal end of the shield towards the waist, and with at least part of the second section tapering outward from the waist towards a distal end of the shield;
at least one passageway extending entirely through the first section and the second section, with the passageway having a minimum cross section at the waist; and
the first section and the second section comprising a flexible material.

22. The shield of claim 21 with the first section having a generally conical shape.

23. The shield of claim 21 wherein the flexible material is transparent or translucent.

24. The shield of claim 21 further comprising a plurality of internal walls forming internal passageways in the shield, and with the internal passageways connected to an irrigation or suction source.

25. The shield of claim 24 further comprising a manifold on the shield connected to the internal passageways and to the source.

26. The shield of claim 21 further comprising one or more lighting elements in the shield.

27. The shield of claim 21 further comprising one or more temporary invaginations in the shield for reducing the diameter of the shield for movement of the shield through an opening to access a surgical site.

28. The shield of claim 21 further comprising a tamping tool having a shaft extending through the passageway, and a sleeve surrounding the shield and holding the shield into a reduced diameter configuration.

29. The shield of claim 21 further comprising a tamping tool having a shaft extending through the passageway, and a delivery tube surrounding the shield and holding the shield into a reduced diameter configuration.
